(19) United States
(12) Patent Application Publication
(10) Pub. No.: US 2007/0100279 A1
(43) Pub. Date: May 3, 2007

(54) Radiopaque-balloon microcatheter and methods of manufacture

(75) Inventor: Mark C. Bates, Charleston, WV (US)

Correspondence Address: LUCE, FORWARD, HAMILTON & SCRIPPS LLP, 11988 EL CAMINO REAL, SUITE 200, SAN DIEGO, CA, 92130, US

(73) Assignee: Paragon Intellectual Properties, LLC, Charleston, WV (US)

(21) Appl. No.: 11/267,226

(22) Filed: Nov. 3, 2005

(51) Int. Cl. A61M 29/00; A61M 25/00; A61M 31/00; A61M 37/00

(52) U.S. Cl. 604/103.1; 604/527; 977/700

(57) ABSTRACT

Microcatheters catheters are provided having balloons incorporating radiopaque nanoparticles. Optionally, carbon nanotubes dispersed within the shaft may be configured to react to electrical stimulation, thereby providing a steerable distal end region on the microcatheter. Methods of making the foregoing microcatheters also are provided.

### FIELD OF THE INVENTION

The present invention relates generally to microcatheters for performing interventional procedures, such as stenting, in fine vessels. More specifically, the present invention relates to a microcatheter made using nanotechnology.

### BACKGROUND OF THE INVENTION

Interventional techniques have been developed wherein catheters are used to perform diagnostic and therapeutic procedures, such as stenting and angioplasty. As the medical field becomes increasingly advanced, there is a growing need for precision instruments and devices. In particular, the extension of diagnostic and treatment modalities has been limited by the inability to access smaller vessels, such as those presented by the cerebrovasculature and distal coronary systems.

In particular, the contrast agents typically used to inflate the balloons of balloon catheters are relatively viscous. At smaller catheter sizes, difficulties arise due to the high hydraulic resistance encountered in inflating the catheter balloon with such contrast agents through an extremely small inflation lumen. This in turn requires the use of either extremely high pressures, presenting a risk of balloon rupture, or the use of a gas to inflate the balloon, which does not provide radiopacity of the inflation event confirming successful balloon expansion. Because determining balloon position is typically critical to the success of most interventional procedures, there is a need for a microcatheter having a radiopaque balloon that avoids the drawbacks of previously-known designs.

In recognition of the foregoing drawback, U.S. Pat. No. 6,786,889 to Musbach, et al. describes a balloon having enhanced radiopacity achieved by texturing the surface of the balloon or providing a radiopaque ink to an exterior or interior surface of the balloon. One disadvantage to the design described in that patent is that the variations in texture may become less observable as the balloon size decreases. Moreover, depending upon the ink employed, the balloon may present biocompatibility issues.

In addition, attempts to reduce the profile of a catheter by reducing the wall thickness can lead to loss of “pushability” of the catheter, i.e., where the catheter has insufficient stiffness to be advanced over a guide wire when pushed from the proximal end. When coupled with the higher pressures required to inflate the balloon when using small lumens, reduced wall thickness also poses the risk of rupture during inflation.

Size limitations encountered in manufacturing small diameter catheters also preclude the use of certain features, such as steerability, which may be of particular use, for example, in the placement of intracranial embolism coils for treatment of aneurysms. Previously-known commercially available steerable catheters have relatively large diameters due to the presence of control wires or other steering elements, and thus are unsuitable for use in smaller vessels. There is accordingly a need for steerable catheters capable of accessing small vessels, such as intracranial vessels.

In view of the foregoing, it would be desirable to provide a radiopaque balloon for use in a microcatheter that avoids the use of a liquid contrast agent for balloon inflation.

It further would be desirable to provide a microcatheter having a radiopaque balloon that could be inflated with an inert gas at relatively low pressures, thereby avoiding the risk of balloon rupture.

It also would be desirable to reinforce the wall of a microcatheter to enhance pushability and minimize the risk of rupture when using reduced wall thicknesses, while still preserving the longitudinal flexibility of the device.

It still further would be desirable to provide a steerable catheter, suitable for use in smaller vessels, wherein the steering mechanism does not substantially increase the diameter of the catheter.

### SUMMARY OF THE INVENTION

In view of the foregoing, it is an object of the present invention to provide a radiopaque balloon for use in a microcatheter that avoids the use of a liquid contrast agent for balloon inflation.

It also is an object of this invention to provide a microcatheter having a radiopaque balloon that could be inflated with an inert gas at relatively low pressures, thereby avoiding the risk of balloon rupture.

It is another object of this invention to provide a microcatheter having a reinforced wall to enhance pushability and minimize the risk of rupture when using reduced wall thicknesses while still preserving the longitudinal flexibility of the device.

It is a further object of the present invention to provide a steerable catheter, suitable for use in smaller vessels, wherein the steering mechanism does not substantially increase the diameter of the catheter.

These and other objects of the present invention are accomplished by providing a balloon catheter wherein the balloon is manufactured using a material containing radiopaque nanoparticles. As used in this specification, a nanoparticle is particle having an average diameter of less than about 500 nanometers, which may be incorporated into and dispersed within the matrix of a polymer without substantially affecting the mechanical properties of the polymer. Preferably, the nanoparticles comprise gold, platinum, tantalum, palladium, tungsten or an alloy thereof, capable of absorbing X-rays, and therefore being visible when viewed with a fluoroscope.

Advantageously, a particle having an average diameter of 500 nanometers is substantially smaller than commercially-available metallic particles, such as tungsten powder with a diameter of 1 um, which may be twice as large, or bismuth trioxide with a diameter of 9 um, which may be nearly twenty times larger. Those larger particles may compromise the integrity of a thin-walled structure, and are therefore inappropriate to provide radiopacity of a balloon used in certain medical applications.

In addition, the catheter of the present invention preferably includes a catheter shaft that is reinforced with carbon nanotubes, thereby permitting reduced wall thickness while preserving pushability of the catheter. As used herein, nanotubes may be single wall nanotubes, multiwall carbon nanotubes, nanoropes, nanofibers, or similar composition as known in the art of nanotechnology.

In an alternative embodiment, the carbon nanotubes may be arranged in layers within the wall of a distal portion of the catheter, so the catheter alters shape responsive to the application of an electric potential. In this manner, the catheter may be steered without the need for wires or other mechanisms found in previously-known steerable catheters. The balloon catheter of the present invention accordingly may be manufactured with very small profiles, e.g., down to less than 2.5 French, and thus appropriate for use in very small vessels.

Methods of manufacturing the catheter of the present invention also are provided. Radiopaque nanoparticles preferably are obtained and added to a polymer that has been heated above its melting point. The mixture then is agitated to uniformly disperse the nanoparticles. The polymer may then be processed in accordance with balloon molding techniques that are per se known.

Radiopaque nanoparticles suitable for use in the catheter of the present invention may be created by impinging high energy particles against an ingot of source material to release nanoparticles. The nanoparticles then may be collected and characterized, e.g., by size, for specific applications. Alternatively, the nanoparticles may be directly embedded into a suitable balloon material. For example, a balloon material may be disposed within the interior of a chamber. An ingot of a suitable radiopaque source material may then be impacted by high energy particles to release nanoparticles that adhere or become embedded with the balloon material to render the balloon material radiopaque.

The catheter shaft may be formed by mixing pre-formed carbon nanotubes within the catheter polymer, and then extruding the resulting mixture using known techniques. In addition, the wall of the catheter shaft may be co-extruded in multiple passes with intervening layers of arranged to alter the shape of the distal portion responsive to an electrical stimulus. In particular, the catheter may be formed having at least portions of polymer including oppositely-charged nanotubes separated by an electrical insulator. The portions may be disposed concentrically or formed as separate halves of the catheter and then joined along an electrically insulating seam.

Using the principles of the present invention, the precise location of a balloon mounted on a catheter and placed within a patient may be obtained utilizing the radiopaque qualities of the nanoparticles. Additionally, use of a catheter formed with nanotubes in conjunction with a balloon impregnated with nanoparticles may allow for extremely small balloon microcatheters that may be appropriate for use in intracranial applications.

Methods of forming the catheters of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIGS. 1A and 1B are, respectively, a perspective view and side sectional view of an exemplary balloon catheter of the present invention;

FIG. 2 is a side sectional view of a balloon employed in an alternative embodiment of the catheter of the present invention;

FIG. 3 is a flow chart depicting the steps of forming a balloon polymer impregnated with radiopaque nanoparticles;

FIG. 4 is a flow chart depicting alternative steps for forming a radiopaque balloon in accordance with the present invention;

FIG. 5 is a flow chart depicting the steps of forming a catheter shaft reinforced using carbon nanotubes;

FIGS. 6A, 6B, 6C, and 6D are, respectively, a perspective view, a cross-sectional view, another cross-sectional view, and an enlarged perspective view of an alternative embodiment of a balloon catheter of the present invention; and

FIG. 7 is a flow chart depicting steps for forming the steerable microcatheter of FIGS. 6 using nanotubes in accordance with the present invention.

### DETAILED DESCRIPTION OF THE INVENTION

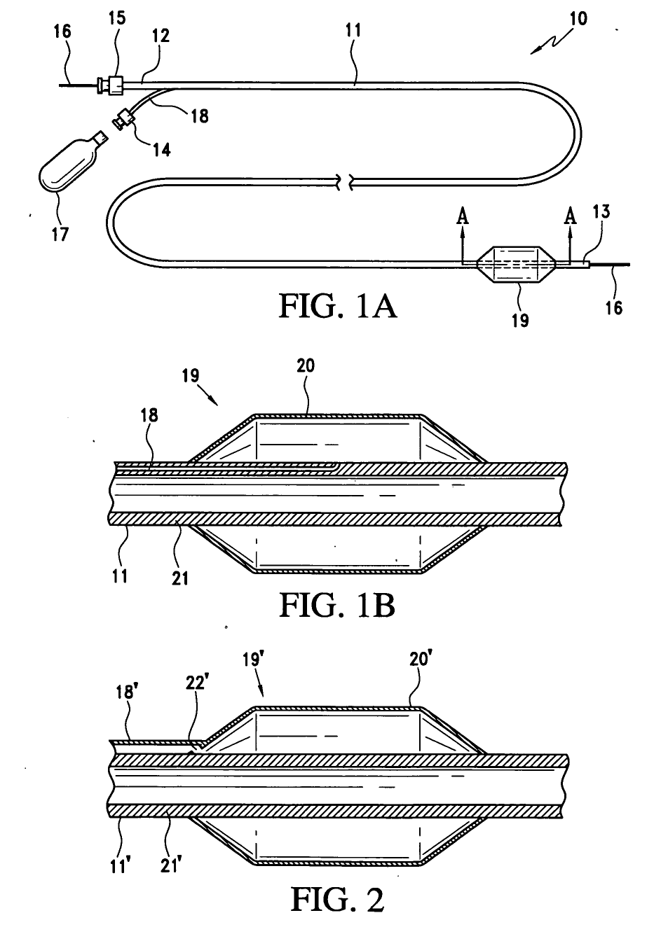

Referring to FIGS. 1A and 1B, a microcatheter constructed in accordance with the principles of the present invention is described. Microcatheter 10 comprises shaft 11, proximal end 12, distal end 13, inflation port 14, manifold 15, guide wire 16, fluid source 17, inflation lumen 18, and balloon 19.

Fluid source 17, preferably pressurized gas, such as carbon dioxide (CO2), helium, air, or other fluid, may be attached directly to inflation port 14, or may alternatively pass first through a regulation device to control pressure, flow rate, or other fluid properties. Fluid source 17 is in fluid communication with balloon 19 through inflation lumen 18. Fluid source 17 preferably comprises a CO2 tank having a regulator to control inflation of balloon 19 via inflation port 64.

In FIG. 1B, balloon 19 comprises flexible member 20 affixed to shaft 11. Inflation lumen 18 passes through wall 21 of shaft 11, so that a distal end of inflation lumen 18 communicates with the space defined between the outer surface of shaft 11 and the inner surface of flexible member 20. The proximal end of inflation lumen 18 is coupled to inflation port 14.

Balloon 19 differs from previously-known balloons by having nanoparticles embedded or disposed in flexible layer 20. Nanoparticles preferably are disposed substantially uniformly throughout flexible layer 20, in a manner discussed more thoroughly below. The presence of the x-ray absorbent nanoparticles allows the balloon to be visualized using fluoroscopic imaging techniques.

Microcatheter 10 also may comprise carbon nanotubes disposed within wall 21. The nanotubes provide increased axial strength and pushability, thereby reducing the likelihood of kinking, binding, or similar problems encountered when the wall thickness of a catheter shaft is reduced. It should be understood that microcatheter 10, although shown here as an over-the-wire design, also could be made in a rapid exchange configuration.

In an alternative embodiment of the balloon 20, designated 20′ in FIG. 2, inflation lumen 18′ is disposed against wall 21′ of shaft 11′. Port 22′ exists in flexible member 20′, and inflation lumen 18′ may pass through port 22′ and terminate within balloon 19′. Alternatively, the distal end of inflation lumen 18′ may be affixed to flexible member 20′ such that it provides communication through port 22′.

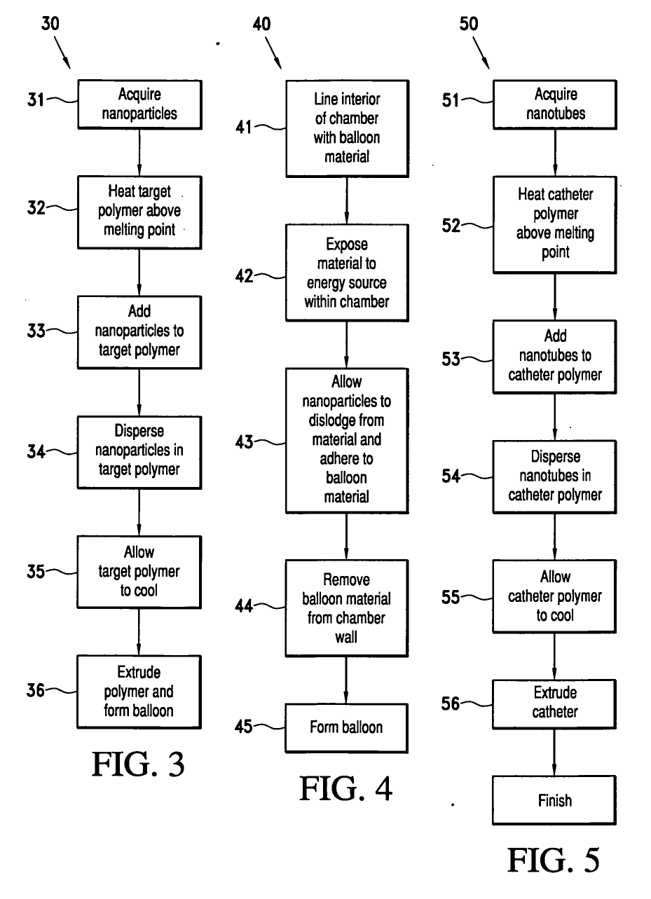

Referring now to FIG. 3, a preferred method of forming a balloon having nanoparticles disposed within it is described. At step 31, nanoparticles may be acquired from commercially available sources or may be created in a laboratory setting. One method of creating suitable nanoparticles is to place a piece of source material into a collection chamber. The source material may be an ingot of gold, platinum, silver, palladium, tungsten, or other radiopaque material known in the art. The source material then is exposed to a laser or other energy source to release nanoparticles of the source material, which adhere to the interior of the collection chamber. Once the collection chamber is cooled, the nanoparticles are collected by scraping the interior of the collection chamber.

Next, at step 32, the balloon polymer is heated above its melting point. Suitable balloon polymers may include polyester (PET), nylon, polyurethane, polyvinyl chloride (PVC), polyethylene terephalate (PET) or other polymer that is biocompatible and appropriate for use as a balloon.

At step 33, the nanoparticles are mixed into the melted balloon polymer while the polymer is maintained at a temperature above its melting point. At step 34, the mixture is mechanically agitated to thoroughly distribute the nanoparticles in the polymer. This may be accomplished using an ultrasonic homogenizer, but also may be accomplished by using a material compounder, moving the container holding the catheter polymer/nanoparticle solution, stirring the catheter polymer/nanoparticle solution, or by maintaining the catheter polymer/nanoparticle solution above the melting temperature for an extended period of time.

Once the nanoparticles are distributed to a satisfactory degree, the balloon polymer/nanoparticle solution is allowed to cool, at step 35. Finally, at. step 36, the balloon is formed using conventional balloon molding techniques. It should be understood that prior to forming the balloon, the polymer/nanoparticle solution optionally may be pelletized and re-extruded or formed using known techniques.

Referring now to FIG. 4, alternate method 40 of creating a radiopaque balloon is described. In this method, the interior of a collection chamber is lined with balloon material at step 41. For example, a sheet of balloon material such as polyester (PET), nylon, polyurethane, polyvinyl chloride (PVC), polyethylene terephalate (PET) or other polymer that is biocompatible and appropriate for use as a balloon, may be disposed within the interior of the chamber.

At step 42, a piece of source material is placed into the collection chamber. The source material may be gold, platinum, silver, palladium, tungsten, or other radiopaque material. The source material then is exposed to an energy source to release particles directly onto the balloon material. Energy source may include laser; plasma deposition, or ion beam deposition. Due to the exposure to the energy source, nanoparticles will be released from the source material and preferably will become deposited on or embedded within the sheet of polymer at step 43. The energy source then may be discontinued and the balloon material removed at step 44. At step 45, the balloon is formed using conventional balloon molding techniques.

FIG. 5 depicts method 50 of making the microcatheter of FIGS. 1 in accordance with the present invention. At step 51, carbon nanotubes are acquired from a commercial source or made using known techniques. As used herein, nanotubes may be single wall nanotubes, multiwall carbon nanotubes, nanoropes, or nanofibers.

At step 52, a catheter polymer is heated above its melting point. Suitable catheter polymers include polyester (PET), polyolefin, fluoropolymers (PTFE), polyvinyl chloride (PVC), polyethylene, urethanes, and polyethylene terephalate (PET). At step 53, the nanotubes are mixed into the melted catheter polymer while the polymer preferably is maintained at a temperature above its melting point.

At step 54, the nanotubes are dispersed within the polymer, for example, by agitating the mixture. This may be accomplished using an ultrasonic homogenizer, but also may be accomplished by using a material compounder, moving the container holding the catheter polymer/nanotube solution, stirring the catheter polymer/nanotube solution, or by maintaining the catheter polymer/nanotube solution above the melting temperature for an extended period of time. It should be understood that the use of solvents or other additives also may be appropriate for dip molding or other procedures.

At step 55, the catheter polymer/nanotube mixture is allowed to cool to a temperature that facilitates extrusion of the catheter. At step 56, the catheter is extruded using known techniques. It should be understood that prior to forming the catheter, the polymer/nanotube solution optionally may be pelletized and re-extruded or formed using known techniques.

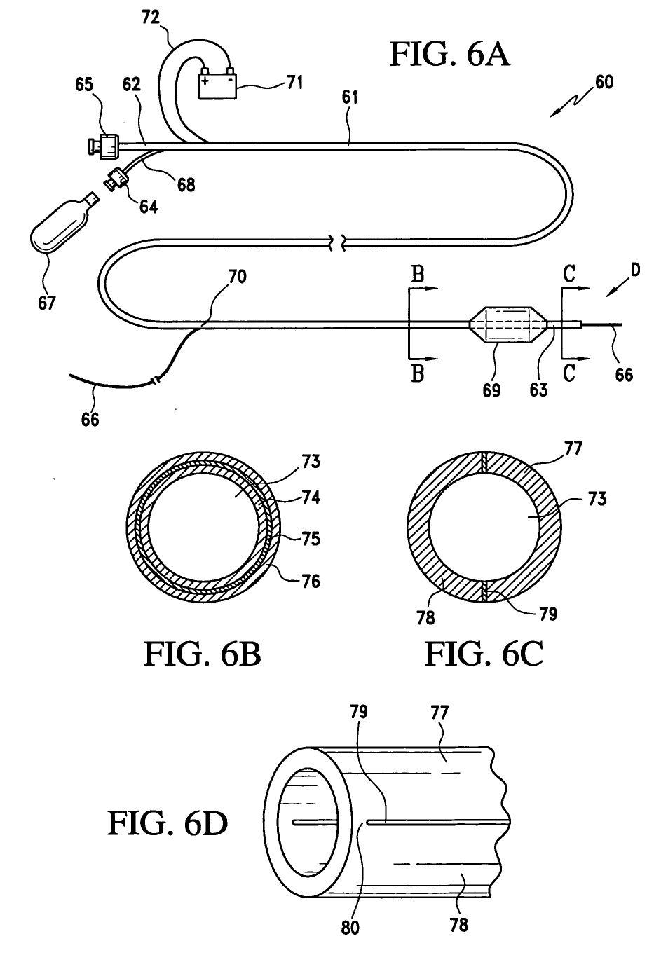

With respect to FIGS. 6A, 6B, 6C and 6D, steerable microcatheter 60 is described. Microcatheter 60 has a rapid-exchange design, although it should be understood that the principles of the present invention apply equally to an over-the-wire design. Microcatheter 60 incorporates many features typical of a rapid exchange catheter, such as shaft 61, proximal end 62, distal end 63, inflation port 64, manifold 65, guide wire 66, fluid source 67, inflation lumen 68, balloon 69, and skive 70.

Fluid source 67 is comparable to fluid source 17 as described above, and accordingly is in fluid communication with balloon 69 through inflation lumen 68. Fluid source 67 preferably comprises CO2 tank 67 having a regulator to control inflation of the balloon through inflation port 64.

Microcatheter 60 also comprises power source 71, which may be internal or external to the catheter system, to provide a direct current via wires 72. In a preferred embodiment, power source 71 is an external battery and is coupled to wires 72 via couplers. Wires 72 preferably are electrically insulated to allow the transmission of a DC current or voltage. Wires 72 are attached to charged portions of microcatheter 60, allowing steerability, as described hereinbelow.

Steerability of microcatheter 60 is provided by applying a voltage potential across portions of microcatheter 60 having different intrinsic charges. Flexion and extension of a planar member having positively and negatively charged portions is described in Ray H. Baughman et al., Carbon Nanotube Actuators, SCIENCE, May 21, 1999, at 1340-1344, which is hereby incorporated by reference in its entirety. In FIGS. 6, charged portions of microcatheter 60 are arranged as concentric annuli and, at distal end 63, as semicylindrical portions joined to define lumen 73. Portions of microcatheter 60 having opposite charges contact one another at distal end 63, as depicted in FIG. 6D, but otherwise are insulated from each other. Thus, movement of microcatheter 60 in response to electrical stimulation occurs in primarily in the vicinity of distal end 63, as described below.

More specifically, FIG. 6B provides a cross-sectional view of microcatheter 60 taken along line B-B in FIG. 6A, whereas FIG. 6C provides a cross-sectional view taken along line C-C in FIG. 6A. In a preferred embodiment, the transition from the cross-sections shown in FIGS. 6B and 6C occurs distal to balloon 69. FIG. 6D depicts the junction of oppositely charged polymer portions from the perspective of an observer looking in direction D.

In FIG. 6B, concentric layers of charged polymer are shown, in which inner layer 74 comprises a catheter polymer that has been treated with charged nanotubes. Insulator 75 is disposed surrounding inner layer 74 and may comprise a non-conductive coating of polymer of other material which preferably is thin, biocompatible, and pliable. Outer layer 76 surrounds insulator 75 and comprises a catheter polymer that has been treated with charged nanotubes that are opposite in charge to those used in inner layer 74. Lumen 73 passes through the center of microcatheter 60; guide wire 66, inflation lumen 68, and other details are omitted for clarity.

In FIG. 6C, a distal portion of microcatheter 60 comprises charged portions having a semicylindrical shape and which deflect in response to electrical stimuli. Preferably, the charged polymers are semicylindrical in only a relatively short length near the distal tip of the catheter. In contrast, no flexion or extension is desired along the length of catheter having the concentric annuli cross-section of FIG. 6B. Positively charged polymer 77 is separated from negatively charged polymer 78 by insulator 79. Lumen 73 is passes through the center of microcatheter 60; guide wire 66 and other details are omitted for clarity.

Referring now to FIG. 6D, positively charged polymer 77 is in communication with negatively charged polymer 78 at junction 80. Accordingly, at this location an electrical current may pass from a source, through positively charged polymer 77, junction 80, and negatively charged polymer 78. Power source 71 is connected to positively charged polymer 77 and negatively charged polymer 78 by wires 72 so that these components comprise a portion of a circuit that includes junction 80.

Preferably, balloon 69 is disposed proximal of the steerable distal section. For example, the steerable portion of microcatheter 60 has the cross-section depicted in FIG. 6C, preferably distal of balloon 69, whereas balloon 69 preferably is affixed to shaft 61 at a location having the cross-section shown in FIG. 6B. Such positioning reduces the likelihood that balloon 69 will move or dislodge upon movement of the steerable section. Nevertheless, some applications may benefit from a catheter having a balloon distal to a flexible section or even along a flexible section, and those embodiments are recognized as within the scope of the present invention.

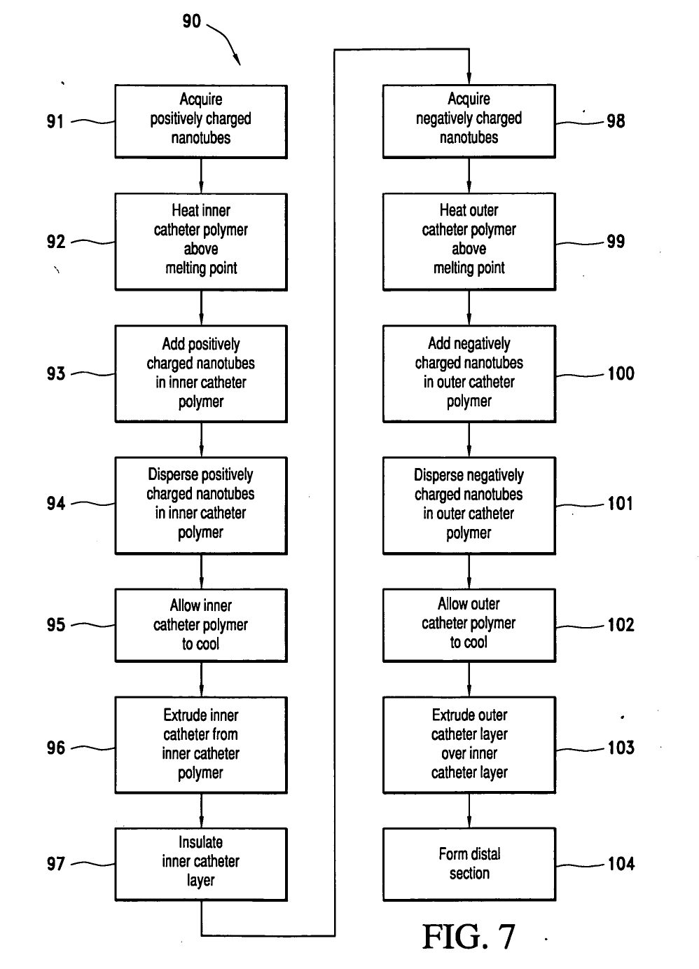

Referring now to FIG. 7, a method of making steerable catheter 60 is described. At step 91, positively charged nanotubes are obtained. The nanotubes may be constructed using known techniques or acquired from a commercial source. Nanotubes may acquire a charge by undergoing charge injection as described in Ray H. Baughman et al., Carbon Nanotube Actuators, SCIENCE, May 21, 1999, at 1340-1344. Using-such a method, positively charged nanotubes and negatively charged nanotubes may be prepared.

In the following description, a method is described for making a catheter having a positively charged inner layer and a negatively charged outer layer. It should of course be understood that the method applies equally to a catheter having a negatively charged inner layer and a positively charged outer layer.

At step 92, the catheter polymer is heated above its melting point. Suitable catheter polymers include, for example, polyester (PET), polyolefin, fluoropolymers (PTFE), polyvinyl chloride (PVC), polyethylene, urethanes, and polyethylene terephalate (PET). At step 93, positively charged nanotubes are mixed into the melted catheter polymer while the polymer is maintained at a temperature above its melting point.

At step 94, the mixture is agitated to disperse the positively charged nanotubes in the inner catheter polymer. This step may be accomplished using apparatus as described herein above.

At step 95, the catheter polymer/nanotube solution is allowed to cool to a temperature that facilitates extrusion of the catheter. The inner layer of the catheter then is extruded at step 96, using techniques that are per se known in the art of catheter construction.

Once extruded, the an insulating barrier or coating is applied to the inner catheter layer at step 97. This barrier may be a non-conductive coating of polymer of other material which preferably is thin, biocompatible, and pliable.

At step 98, the negatively charged nanotubes are acquired. At step 99, the polymer for the outer catheter layer is heated above its melting temperature and the negatively charged nanotubes are mixed into the melted outer catheter polymer at step 100. At step 101, the mixture is agitated to promote thorough distribution of the negatively charged nanotubes, using the techniques described above for step 94.

At step 102, the catheter polymer/nanotube solution is allowed to cool to a temperature that facilitates extrusion of the catheter. At step 103, the outer layer of the catheter is extruded over the insulated inner layer to form a coaxial covering.

Finally, at step 104, the distal section is shaped and attached to a portion of layered catheter. On one side of the distal section, the catheter polymer has been treated with negatively charged nanotubes, whereas the other side of the distal section has polymer treated with negatively charged nanotubes. Aside from an area of communication at the distalmost end, referred to as junction 80 above, these two sides are insulated from each other. Moreover, the two sides are in electrical communication with the similarly charged inner and outer layers of the catheter.

It should be understood that a catheter having increased strength and pushability may be formed by the addition of nanotubes to a catheter polymer without the need to obtain oppositely charged nanotubes. This type of non-steerable catheter would be appropriate for use with the design of microcatheter 10 described above and shown in FIGS. 1, and could be formed in a similar manner as described in steps 91 to 96, with the exclusion of any reference to charges or inner/outer catheter portions.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. A catheter comprising:
an elongated shaft having proximal and distal ends and a lumen therebetween; and
a balloon affixed to the elongated shaft near the distal end, the balloon comprising a polymer in which a multiplicity of radiopaque nanoparticles is dispersed.

2. The apparatus of claim 1 wherein the radiopaque nanoparticles are selected from the group consisting of gold, platinum, silver, palladium, tungsten and tantalum.

3. The apparatus of claim 1 wherein the elongated shaft further comprises a polymer having a multiplicity of carbon nanotubes disposed therein to enhance pushability of the catheter.

4. The apparatus of claim 3 further comprising charged nanotubes disposed within the body of the elongated shaft and arranged to respond to electrical stimulation.

5. The apparatus of claim 4 further comprising a source of electrical stimulation.

6. The apparatus of claim 5 wherein the elongated shaft further comprises a first portion comprising positively charged nanotubes and a second portion comprising negatively charged nanotubes.

7. The apparatus of claim 6 further comprising an insulator disposed between the first portion and the second portion.

8. The apparatus of claim 6 wherein the first portion is in communication with the second portion at a junction near the distal end of the elongated shaft.

9. The apparatus of claim 8 wherein the source of electrical stimulation communicated with the first portion and second portion via wires.

10. The apparatus of claim 9 wherein the source of electrical stimulation is a battery.

11. The apparatus of claim 1 wherein the catheter further comprises a balloon inflation lumen coupled between the balloon and the proximal end of the elongated shaft.

12. The apparatus of claim 11 further comprising a compressed gas container coupled to the inflation lumen.

13. A balloon catheter comprising:
an elongated shaft having proximal and distal ends and a lumen therebetween; and
a balloon having an interior, the balloon affixed to distal end of the elongated shaft;
wherein the balloon comprises a polymer selected from the group consisting of polyester, polyolefin, fluoropolymers, polyvinyl chloride, polyethylene, urethanes, and polyethylene terephalate, and a multiplicity of nanoparticles disposed within the polymer.

14. The apparatus of claim 13 further comprising an inflation lumen having a distal end and a proximal end, the distal end in fluid communication with the interior of the balloon and the proximal end disposed at a location substantially near the proximal end of the shaft.

15. The apparatus of claim 14 further comprising an inflation port disposed at the proximal end of the inflation lumen.

16. The apparatus of claim 13 further comprising a rapid-exchange port disposed in a lateral wall of the shaft.

17. The apparatus of claim 13 wherein the shaft further comprises nanotubes disposed within the body of the shaft.

18. The apparatus of claim 17 wherein the nanotubes comprise positively charged nanotubes and negatively charged nanotubes.

19. The apparatus of claim 18 wherein the catheter is steerable in response to electrical stimulation.

20. A method of forming a catheter component comprising:
providing a plurality of nanoparticles or nanotubes and a polymer having a melting point;
heating the polymer above its melting point;
adding the plurality of nanoparticles or nanotubes to the polymer;
dispersing the plurality of nanoparticles or nanotubes within the polymer;
cooling the polymer; and
forming the polymer into a catheter component.

21. The method of claim 20 in which dispersing the plurality of nanoparticles or nanotubes within the polymer comprises agitating the polymer.

22. The method of claim 21 in which dispersing the plurality of nanoparticles or nanotubes within the polymer comprises using an ultrasonic homogenizer.

23. The method of claim 21 further comprising maintaining the polymer above its melting point for a period of time during dispersing the plurality of nanoparticles or nanotubes within the polymer.

24. The method of claim 20 wherein the nanoparticles are radiopaque and the catheter component comprises a balloon.

25. The method of claim 20 wherein the nanotubes are carbon nanotubes and the catheter component comprises a catheter shaft.

26. The method of claim 25 wherein carbon nanotubes have first and second electrical charges, and forming the catheter component further comprises:
extruding an inner layer from polymer containing carbon nanotubes having the first electrical charge;
affixing a layer of electrical insulation to an exterior of the inner layer; and
extruding over the layer of electrical insulation an outer layer from polymer containing carbon nanotubes having the second electrical charge.
